(19) United States
(12) Patent Application Publication
(10) Pub. No.: US 2023/0368352 A1
(43) Pub. Date: Nov. 16, 2023

(54) SYSTEM AND METHODS FOR QUANTIFICATION OF SUBSTANCE CONCENTRATION IN BODY STRUCTURES USING SPECTRAL COMPUTED TOMOGRAPHY

(71) Applicant: VARIAN MEDICAL SYSTEMS, INC., Palo Alto, CA (US)

(72) Inventors: Bernd HOFMANN, Erlangen (DE); Matthias LICHY, Nürnberg (DE); Bernhard SCHMIDT, Fürth (DE)

(21) Appl. No.: 18/317,696

(22) Filed: May 15, 2023

Related U.S. Application Data
(60) Provisional application No. 63/342,503, filed on May 16, 2022.

(51) Int. Cl. G06T 7/00; A61K 51/12

(52) U.S. Cl. CPC G06T 7/00; A61K 51/1251; A61K 49/0423

(57) ABSTRACT

A method of determining a concentration of a chemical substance in a body of a patient includes adding an additive quantity of an additive material to a base quantity of a chemical substance to obtain a mixed composition having a predetermined density and delivering the mixed composition to the body of the patient. The method also includes obtaining an image of at least a portion of the body of the patient and determining a concentration of the chemical substance in the at least a portion of the body of the patient based on the image and the predetermined density of the mixed composition.

### CROSS REFERENCE TO RELATED APPLICATIONS

This application claims the benefit of U.S. Provisional Patent Application No. 63/342,503, filed May 16, 2022, which is hereby incorporated by reference for all purposes as if fully set forth herein.

### FIELD

The present disclosure relates to methods for the quantification of substance concentration in body structures using spectral computed tomography (CT).

### BACKGROUND

This section provides background information related to the present disclosure which is not necessarily prior art.

Chemical substances such a therapeutic substances medicines are delivered to the body of a patient for various treatments and diagnostic purposes. Such chemical substances can be delivered into certain targeted areas of the body of patient such as target regions, organs or pathologies. It is desirable to deliver the chemical substance to the target location in sufficient and/or known concentrations to deliver a particular therapeutic or diagnostic result. Incorrect or insufficient concentrations may lead to ineffective treatments or results. The delivery of excessive or elevated concentrations can lead to harmful effects to healthy tissues in the body of the patient or to inconclusive diagnostic results.

Existing and traditional method of determining concentrations of chemical substances suffer from many drawbacks. Existing and traditional methods may produce inaccurate or qualitative concentration determinations. In addition, existing and traditional methods may not be used in connection with some therapeutic or diagnostic procedures. There exists a need, therefore, for improved methods for determining the concentration of chemical substances in the bodies of patients.

### SUMMARY

This section provides a general summary of the disclosure, and is not a comprehensive disclosure of its full scope or all of its features.

In some embodiments of the present disclosure, a system and methods are provided that allow the quantification of the concentration of chemical substance in the body of a patient. The chemical substance can include various substances such as therapeutic substances, chemotherapy substances, medicines, diagnostic substances and the like. The chemical substance can be delivered in various forms such as liquids, microspheres, embolic microspheres, radioembolic microspheres, nanoparticles or the like. The method of the present disclosure allows a quantitative determination of the concentration of the chemical substance using a labelling or marking material and the use of spectral or dual energy computed tomography (CT) imaging.

In some embodiments of the present disclosure, a method of determining a concentration of a chemical substance in a body of a patient includes introducing a marking additive to a chemical substance for introduction into a body of a patient and obtaining an image of at least a portion of the body of the patient using a spectral computed tomography (CT) imaging device. The method can also include determining a concentration of the chemical substance in the body based on the image.

In some embodiments of the present disclosure, a method of determining a concentration of therapeutic particles in a body of a patient includes adding a marking quantity of marking particles to a therapeutic quantity of therapeutic particles to obtain a mixed quantity of particles. The marking particles are detectable by a spectral computed tomography (CT) imaging device. The method can also include delivering the mixed quantity of particles to the body of the patient, obtaining an image of at least a portion of the body of the patient using the CT imaging device, and determining a concentration of the therapeutic particles in the body based on the image.

In some embodiments of the present disclosure, a method of determining a concentration of a chemical substance in a body of a patient includes adding an additive quantity of an additive material to a base quantity of a chemical substance to obtain a mixed composition having a predetermined density. The method can also include delivering the mixed composition to the body of the patient, obtaining an image of at least a portion of the body of the patient, and determining a concentration of the chemical substance in the at least a portion of the body of the patient based on the image and the predetermined density of the mixed composition.

In some embodiments of the present disclosure, a system includes a source including a chemical substance and a marking additive that is spectrally visible; an imaging device; and a computing device configured to analyze an image captured by the imaging device of a tissue after the chemical substance and the marking additive have been delivered to the tissue and determine a concentration of the chemical substance in the tissue.

In an aspect, the chemical substance is therapeutic and configured to deliver a drug or other therapeutic chemical to a target region in the tissue.

In an aspect, the marking additive is one of iodine and gadolinium. In an aspect, the marking additive is incorporated into the chemical substance.

In an aspect, the chemical substance and the marking additive flow and deposit homogeneously in vasculature of the tissue.

In an aspect, a ratio of the chemical substance to the marking additive is known and the concentration of the chemical substance is determined by converting a concentration of marking additive to the concentration of the chemical substance based on the ratio.

In an aspect, a density of a mixture of the chemical substance and the marking additive is known and the concentration of the chemical substance is determined by based on the density.

In an aspect, the imaging device is a spectral computed tomography (CT) device.

In some embodiments of the present disclosure, a method of determining a concentration of a chemical substance in a body of a patient includes obtaining an image of a portion of the body of the patient using a spectral computed tomography (CT) imaging device after a marking additive and the chemical substance have been introduced into the portion of the body of the patient; and determining a concentration of the chemical substance in the portion of the body of the patient based on the image.

In an aspect, the marking additive is applied as a coating to the chemical substance.

In an aspect, the concentration of the chemical substance is determined by directly observing and measuring the concentration of the chemical substance.

In an aspect, the chemical substance and the marking additive are mixed to obtain a mixed composition having a predetermined density, and the concentration of the chemical substance is determined based on the image and the predetermined density of the mixed composition.

In an aspect, the chemical substance is one of a liquid, microspheres, embolic microspheres, radioembolic microspheres, and nanoparticles.

In an aspect, the chemical substance and the marking additive are introduced into the body of the patient by subcutaneous, intramuscular, or intravenous injection.

In some embodiments of the present disclosure, a method of determining a concentration of delivery particles in a body of a patient includes adding a first quantity of marking particles to a second quantity of delivery particles to obtain a mixed quantity of particles, the marking particles detectable by a spectral computed tomography (CT) imaging device; delivering the mixed quantity of particles to the body of the patient; obtaining an image of a portion of the body of the patient using the CT imaging device; and determining a concentration of the delivery particles in the body based on the image.

In some embodiments of the present disclosure, use of a mixed composition of an additive material and a chemical substance in a predetermined ratio as a detectable composition within a patient body by an imaging device to determine a concentration of the chemical substance in a portion of the patient body based on an image from the imaging device and the predetermined ratio of the mixed composition.

In an aspect, the additive material includes a spectrally detectable material such as iodine or gadolinium.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a diagram illustrating an example treatment system in accordance with some embodiments of the present disclosure.

FIG. 2 is a flow chart illustrating an example method of determining a concentration of a therapeutic substance in a body of a patient in accordance with some embodiments of the present disclosure.

FIG. 3 is a flow chart illustrating an example method of determining a concentration of therapeutic particles in a body of a patient in accordance with some embodiments of the present disclosure.

FIG. 4 is a flow chart illustrating an example method of determining a concentration of a chemical substance in a body of a patient in accordance with some embodiments of the present disclosure.

Corresponding reference numerals indicate corresponding parts throughout the several views of the drawings.

### DETAILED DESCRIPTION

Example embodiments will now be described more fully with reference to the accompanying drawings.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

In various embodiments of the present disclosure, methods of determining a concentration of a chemical substance in the body of a patient are provided. The methods may utilize images obtained using a spectral or dual energy computed tomography (CT) imaging device. Such devices may include the use of two X-ray energy spectra for obtaining imaging information of a body or portion of a body of a patient. Marking or labeling materials may be added or combined with a therapeutic or chemical substance. The marking or labeling material can identify a location and concentration of the chemical substance in the body of a patient. The density of the material(s) delivered to the patient may be used to locate and determine a concentration of the material(s) via a spectral CT image.

The methods of the present disclosure are improvements over existing and traditional methods of determining or assessing the distribution of therapeutic or other materials in the body of a patient. In existing and traditional methods, a contrast agent may be introduced into the body of patient and then imaged to determine a vasculature or other information regarding blood flow of the patient. These methods do not directly determine a concentration or distribution of a therapeutic substance. Instead, an estimation or quantitative determination is made based on the contrast agent. Inaccuracies often result from such procedures. The existing methods are performed in a procedure prior to the actual clinical treatment. Furthermore, the contrast agent may not behave in the body of the patient in the same manner as the therapeutic substance.

In other existing methods, a radioactive tracer may be introduced and imaged using imaging devices that can detect such radioactive tracers such as single-photon emission computerized tomography (SPECT) devices or positron emission tomography (PET) devices. Such methods, however, may have similar disadvantages as those described above and do not provide a direct quantitative determination of concentrations of a therapeutic substance (e.g., a radioembolization microsphere).

The methods of the present disclosure, as described further below, are improvements over these existing or traditional methods by providing direct quantitative determinations of concentrations of chemical substances in the body of a patient undergoing a treatment or diagnostic procedure. The methods of the present disclosure may be employed in a variety of treatments and diagnostic procedures. In various examples, the methods of the present disclosure may be used to determine a concentration of a therapeutic substance delivered via chemotherapy, an embolic microsphere treatment, a radioembolic microsphere treatment, and the like. In various other examples, the methods of the present disclosure may be used in the context of various diagnostic procedures that may be used to determine blood flow and/or blood volume, determine stroke risk and/or stroke treatments, determine tumor vasculature, determine liver deposits, determine kidney extraction, and the like.

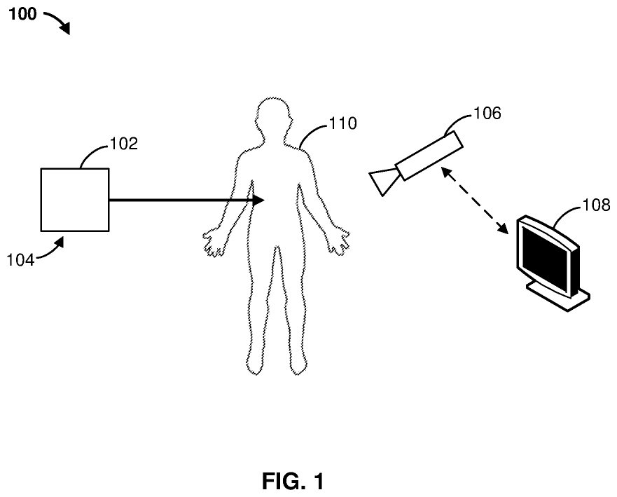

Referring now to FIG. 1, an example treatment system 100 is shown. The treatment system 100 may include one or more devices that are used to perform a treatment or diagnostic process on a patient 110. The system 100 may include a source 102 that can be used to hold a volume or quantity of a chemical substance 104. The chemical substance can be a therapeutic substance such as a medicine, radioactive material, or the like. The chemical substance 104 can be in the form of a liquid, microparticle, microsphere, nanoparticle, or the like. The source 102 can be a suitable vial, syringe, container, bag or other particle. The chemical substance 104 can be delivered to the patient 110 to perform the treatment or procedure.

The system 100 may also include an imaging device 106 and a computing device 108. The imaging device 106 may be a dual energy or spectral computed tomography (CT) imaging device. Any suitable spectral CT imaging device can be used including source-based and detector-based spectral CT imaging devices. The computing device 108 can include a processor, memory and transceiver to allow the computing device to receive and send data, store data (such as executable instructions) and a processor to execute operations. The computing device 108 can be a workstation, desktop computer, laptop, tablet, server, or other suitable computing device.

As will be further explained below, the methods of the present disclosure can be performed using a system such as the treatment system 100 of FIG. 1. In other examples, however, variations of the treatment system 100 can be used or other systems and apparatuses including systems and apparatuses with more than the elements shown in FIG. 1 or in systems and apparatuses including portions of the system 100.

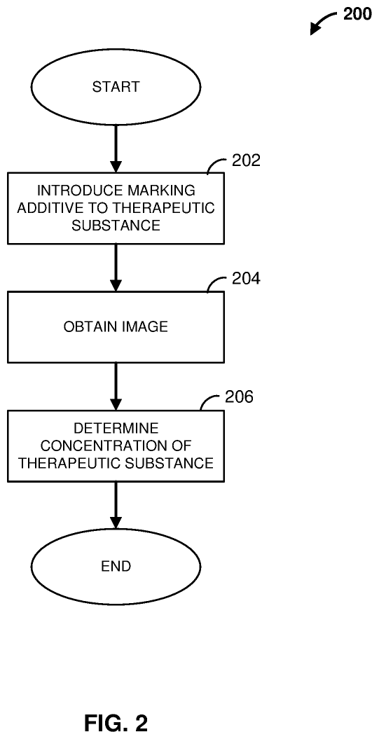

Turning now to FIG. 2, an example method 200 of determining the concentration of a therapeutic substance is shown. The method 200 can be performed using treatment system 100, in some embodiments. In others, other systems and apparatuses can be used. The method 200 begins at step 202. At step 202, a marking additive can be introduced to a chemical substance such as a therapeutic substance. In some examples, the therapeutic substance may be a therapeutic microsphere configured to deliver a drug or other therapeutic chemical to a target region in the patient. The marking additive that is added to the therapeutic substance may be various suitable spectrally detectable materials such as Iodine or Gadolinium. The density of such materials, for example, may make the materials spectrally-visible.

The marking additive can be directly applied to the therapeutic substance. The marking additive can be inherently incorporated into the therapeutic substance by forming microspheres with the marking additive, in some examples. In other examples, the marking additive can be applied as a layer or coating to therapeutic particles, such as to microspheres. In other examples, other processes can be used to incorporate the marking additive into the therapeutic substance.

At step 204, an image of the patient is obtained. The image at step 204 is obtained using a spectral CT imaging device, such as imaging device 106 previously described. The image is typically obtained for region of interest such as to an organ, body tissue, vasculature or other portion of the body of the patient. While not shown in FIG. 2, the therapeutic or other chemical substance was previously delivered to the region of interest of the patient. A suitable catheter, syringe, and/or other delivery equipment can inject the therapeutic substance into the patient prior to the imaging at step 204. In this manner, the image obtained at step 204 can indicate a location, distribution and/or concentration of the therapeutic material since such therapeutic material has been marked with marking additive and is spectrally visible.

At step 206, the concentration of the therapeutic substance can be determined using the image obtained at step 204. Rather than using modeling, prediction, or surrogate measurement, the concentration can be determined by observing and measuring the concentration of the therapeutic material directly. Such determination overcomes and/or improves the shortcomings of existing processes. The determination of concentration of the therapeutic substance is more accurate and can be performed in real-time rather than using pre-treatment or post-treatment procedures.

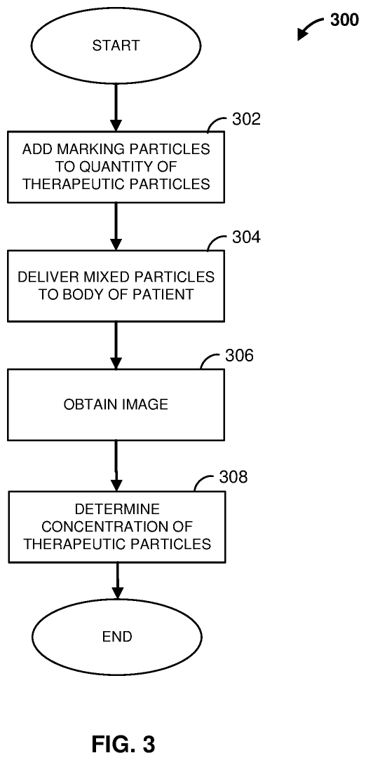

Another example method 300 is shown in FIG. 3. The method 300 provides for determining a concentration of therapeutic particles in the body of a patient. The method 300 may begin at step 302. At step 302, marking particles can be added to a quantity of therapeutic particles. The marking particles may be spectrally-visible such that the marking particles can be detected using a spectral CT imaging device. The marking particles may be various suitable particles. In some examples, the marking particles are chosen based on the type and characteristics of the therapeutic particles being used in a particular treatment for a patient.

In one example, the marking particle can be a particle that biocompatible with the therapeutic particle and includes a spectrally-visible metal such as Iron. The Iron can be incorporated into a microparticle that has similar characteristics to the therapeutic particle. If the therapeutic particle is an embolic microsphere, the marking particle can be prepared or selected to have a similar density, size, shape and other characteristics of the therapeutic particle. The marking particle can be selected so as to have similar flow characteristics of the therapeutic particle so that when the mixture of marking particles and therapeutic particles is delivered to the patient, the mixture flows and deposits homogeneously in the vasculature of the patient. In some examples, the marking particles can be produced to have desired characteristics for use with a particular treatment and complimentary therapeutic particle. In other examples, various marking particles can be produced with various predetermined characteristics and a marking particle can be selected from the various marking particle options.

The marking particles and the therapeutic particles can be mixed in predetermined quantities. In one example, a predetermined marking quantity of the marking particles and a predetermined therapeutic quantity of therapeutic particles can be combined at step 302. With known or predetermined quantities of each type of particle, the mixture defines a known ratio of marking particles to therapeutic particles. The quantities can be measured and combined using weighing, volumetric or other measuring techniques.

At step 304, the mixed particles (i.e., the mixture of marking particles and therapeutic particles) can be delivered to the body of the patient. While not shown in FIG. 3, the mixed particles can be stirred, agitated, shaken or otherwise mixed to increase the homogeneity of the mixture. It is important to have a homogenous mixture to improve the accuracy of the concentration determination that will be performed later in the method. The mixture can be delivered using a syringe, catheter and/or other delivery tools. The mixture can be delivered to predetermined target tissue, tumor, vasculature, or other body tissue.

At step 306, the patient is imaged using a spectral CT imaging device, such as the imaging device 106 previously described. Since the marking particles are spectrally-visible, the marking particles are visible and/or detectable in the image.

At step 308, the concentration of the therapeutic particles is determined. The concentration can be determined by observing and/or detecting the concentration of marking particles and then converting the concentration of marking particles to the concentration of therapeutic particles. The ratio and/or relationship between the marking particles and the therapeutic particles can be known based on the relative quantities of particles combined at step 302. In other examples, modeling of the vasculature and/or experimental testing can be used to determine a relationship between the concentrations of the marking particles and/or the therapeutic particles.

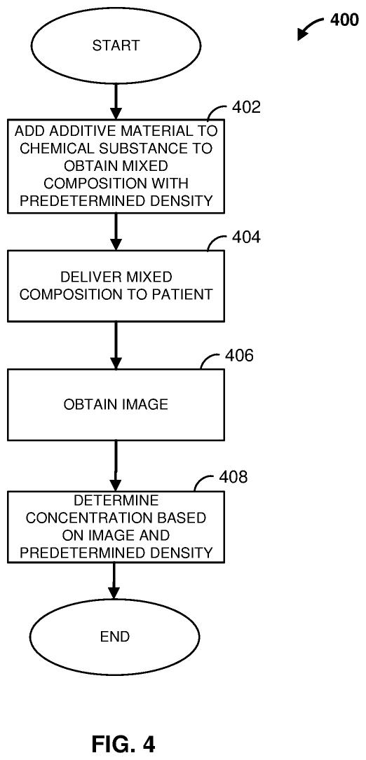

Another example method 400 of the present disclosure is shown in FIG. 4. The method 400 provides an example method for determining a concentration of a chemical substance in the body of a patient. The method 400 may be performed, for example, using the treatment system 100. In other examples, other systems and/or apparatuses can be used.

The method 400 may begin at step 402. At step 402, an additive material may be combined with a chemical substance to obtain a composition with a predetermined density. The chemical substance may be various therapeutic substances or diagnostic substances. The chemical substance may be, for example, a drug, chemotherapy agent, an embolic microsphere containing a therapeutic drug, a diagnostic agent or the like. The additive material may be a second type of particle, microsphere, nanoparticle, fatty material, microbubble or the like.

The density of the chemical substance and the density of the additive material may be known or predetermined prior to the performance of the method 400. In other examples, the additive material may be produced to have a density of a predetermined value. When the additive material is added to the chemical substance, the mixed composition may have a combined density that is known or predetermined. The additive material can be produced and/or configured to make the combined mixture of the chemical substance and the additive material spectrally-visible via a spectral CT imaging device.

The combined mixture of the additive material and the chemical substance can be delivered to the patient at step 404. The mixed composition can be delivered using various delivery tools such as a catheter, syringe, pump, needle or the like. The mixed composition can be delivered to a target tissue such as a tumor, organ, region of the body, or other region of interest.

At step 406, an image can be obtained of the region of interest. The image can be obtained by the imaging device 106. The imaging device can be a spectral CT imaging device. Through the use of the spectral CT imaging device and the known density of the mixture composition, the mixed composition can be detected and/or is spectrally-visible in the image.

At step 408, the concentration of the chemical substance can be determined based on the image obtained at step 406 and the predetermined density of the mixed composition. Because the spectral properties of the mixed composition is known, the concentration of the chemical substance can be determined by observing and/or analyzing the image of the region of interest.

The methods 200, 300, 400 are improvements over existing and traditional methods by providing methods for determining the concentration of chemical substances in the body of a patient with improved accuracy and speed. Since the substances that are delivered to the patient are spectrally visible, the location and distribution of the chemical substances can be directly visible during treatment without the need to wait for post-treatment imaging systems. In addition, the medical professionals performing the treatment can obtain real-time information during treatment to make adjustments, changes or modifications during treatment. These improvements increase the likelihood of an effective treatment.

As discussed above, the use of spectral CT imaging equipment can allow the capture of information characterizing the concentration of a chemical substance in the tissue of a patient. When the densities of the chemical substance and an additive material are known, the concentration of the chemical substance can be determined. Furthermore, spectral CT imaging can be used to monitor and/or determine a perfusion measurement and a concentration achieved during a treatment. After the two-part composition (including a therapeutic substance and a marking substance, e.g., iodine or gadolinium) is delivered to the target tissue, spectral CT imaging can be used to determine a concentration that is delivered to the target tissue and then the concentration of the therapeutic that remains in the tissue. The changes in density in the tissue can be used and imaged using the spectral CT imaging device to determine the achieved concentration and the concentration of therapeutic that remains in the tissue.

The use of the density of the chemical substance and/or the additive material can be used to determine a location and concentration of material in a target tissue of the patient. The methods of the present disclosure can be used to determine a concentration of a therapeutic after the therapeutic is delivered to the patient. In addition, the methods of the present disclosure can also be used to continuously monitor the administration of therapeutic over time. By using region-of-interest (ROI) imaging, a focused aperture can be used to focus imaging energy on a targeted region of interest. The imaging can provide continuous (or semi-continuous) information regarding the concentration of a therapeutic substance in a body tissue. This information can be used to control a dosing of the therapeutic to prevent under or over dosing of the therapeutic. Such a method of treatment can be useful, for example, in the context of chemotherapy.

In addition to providing valuable information for the delivery and administration of therapeutics, this use of a spectral CT scan can also be applied in diagnostic procedures. For example, the methods of the present disclosure can be used to determine a vasculature of various organs such as the brain, lung, liver, kidney and others.

One or more steps of the methods 200, 300, 400 may be performed by a computing device such as computing device 108 (FIG. 1). The computing device 108 may include a concentration determination engine that can determine a concentration of the therapeutic or other chemical substance automatically based on the image obtained from the imaging device 106. Such information can be displayed to a medical professional or other user via a suitable user interface or other reporting tool.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A system comprising:
a source including a chemical substance and a marking additive that is spectrally visible;
an imaging device; and
a computing device configured to analyze an image captured by the imaging device of a tissue after the chemical substance and the marking additive have been delivered to the tissue and determine a concentration of the chemical substance in the tissue.

2. The system of claim 1, wherein the chemical substance is therapeutic and configured to deliver a drug or other therapeutic chemical to a target region in the tissue.

3. The system of claim 1, wherein the marking additive is one of iodine and gadolinium.

4. The system of claim 1, wherein the marking additive is incorporated into the chemical substance.

5. The system of claim 1, wherein the chemical substance and the marking additive flow and deposit homogeneously in vasculature of the tissue.

6. The system of claim 5, wherein a ratio of the chemical substance to the marking additive is known and the concentration of the chemical substance is determined by converting a concentration of marking additive to the concentration of the chemical substance based on the ratio.

7. The system of claim 5, wherein a density of a mixture of the chemical substance and the marking additive is known and the concentration of the chemical substance is determined based on the density.

8. The system of claim 1, wherein the imaging device is a spectral computed tomography (CT) device.

9. A method of determining a concentration of a chemical substance in a body of a patient, the method comprising:
obtaining an image of a portion of the body of the patient using a spectral computed tomography (CT) imaging device after a marking additive and the chemical substance have been introduced into the portion of the body of the patient; and
determining a concentration of the chemical substance in the portion of the body of the patient based on the image.

10. The method of claim 9, wherein the chemical substance is a non-therapeutic substance.

11. The method of claim 9, wherein the marking additive is applied as a coating to the chemical substance.

12. The method of claim 9, wherein the concentration of the chemical substance is determined by directly observing and measuring the concentration of the chemical substance.

13. The method of claim 9, wherein
the chemical substance and the marking additive are mixed to obtain a mixed composition having a predetermined density, and
the concentration of the chemical substance is determined based on the image and the predetermined density of the mixed composition.

14. The method of claim 9, wherein the chemical substance is one of a liquid, microspheres, embolic microspheres, radioembolic microspheres, and nanoparticles.

15. The method of claim 9, wherein the chemical substance and the marking additive are introduced into the body of the patient by subcutaneous, intramuscular, or intravenous injection.

16. A method of determining a concentration of delivery particles in a body of a patient, the method comprising:
adding a first quantity of marking particles to a second quantity of delivery particles to obtain a mixed quantity of particles, the marking particles detectable by a spectral computed tomography (CT) imaging device;
delivering the mixed quantity of particles to the body of the patient;
obtaining an image of a portion of the body of the patient using the CT imaging device; and
determining a concentration of the delivery particles in the body based on the image.

17. The method of claim 16, wherein the delivery particles are a therapeutic substance.

18. The method of claim 16, wherein the marking particles and the delivery particles flow and deposit homogeneously in vasculature of the body of the patient.

19. The method of claim 16, wherein the determining the concentration of the delivery particles in the body is based on a predetermined ratio of the first quantity of marking particles to the second quantity of delivery particles.

20. The method of claim 16, wherein the marking particles include one of iodine and gadolinium.
